(19) United States
(12) Patent Application Publication
(10) Pub. No.: US 2021/0337788 A1
(43) Pub. Date: Nov. 4, 2021

(54) POLYMERIC MICELLES CONTAINING AN ESSENTIAL OIL COMPOUND AND A METHOD OF MAKING SAME

(71) Applicant: JMO Ideas Inc., Toronto (CA)

(72) Inventors: Barkev Keoshkerian, Thornhill (CA); Francesco Merante, Toronto (CA); Siyam Subair, Toronto (CA)

(21) Appl. No.: 17/232,808

(22) Filed: Apr. 16, 2021

Related U.S. Application Data
(60) Provisional application No. 63/012,023, filed on Apr. 17, 2020.

(51) Int. Cl. A01N 25/34; A01N 65/22; A01N 27/00; A01N 31/08; B01J 13/08; B01J 13/14; A61L 2/232; D06M 16/00

(52) U.S. Cl. CPC A01N 25/34; A01N 65/22; A01N 27/00; A01N 31/08; A61L 2101/46; B01J 13/14; A61L 2/232; D06M 16/00; B01J 13/08

(57) ABSTRACT

A method of making an anti-microbial nano-particle containing an essential oil compound (EOC) can include the steps of: a) mixing a quantity of an amphiphilic polymer with a quantity of a solvent to produce a suspension b) heating the suspension to a processing temperature that is higher than a glass transition temperature of the amphiphilic polymer thereby formatting a plurality of polymeric micelles within the solvent, each micelle having a hydrophilic outer portion encasing a hydrophobic core and having a micelle diameter of less than about 80 nm; and c) adding a quantity of an essential oil (EOC) or components of such into the suspension so that a concentration of the essential oil compound is between about 0.2% and about 20% wt, whereby the EOC diffuses into and are encapsulated within the hydrophobic cores of each micelle.

### CROSS-REFERENCE TO RELATED APPLICATION

This application claims the benefit of earlier filed, U.S. provisional application No. 63/012,023 filed Apr. 17, 2020 and entitled Polymeric Micelles Containing An Essential Oil Compound And A Method Of Making Same, the entirety of which is incorporated herein by reference.

### FIELD OF THE INVENTION

In one of its aspects, the present disclosure relates to the production and use of nano-particle, polymeric micelles that can encapsulate an anti-microbial essential oil compound and can help impart antimicrobial properties to surfaces.

### INTRODUCTION

U.S. patent publication no. 2008/0153980 discloses a process for preparing polymer particles, consisting of a latex polymer dispersion including particles of liquid dispersible starting polymer in a dispersion liquid and growing a polymer shell on the particles via a starve fed free radical polymerization process.

U.S. patent publication no. 2008/0210124 discloses nano-sized particles having a core portion comprising a crystalline polymer and a shell portion comprising a polymer derived from at least one monomer not miscible with the crystalline polymer of the shell portion.

U.S. Pat. No. 9,974,753 discloses nanoparticles for encapsulating compounds, the preparation and uses thereof, said nanoparticles being based on a vegetable hydrophobic protein, particularly zein, and a water miscible non-volatile organic solvent, particularly propylene glycol. Said nanoparticles can encapsulate or incorporate a product of interest for use in the agricultural, cosmetic, food or pharmaceutical fields.

Chinese patent publication no. 109260028 relates to relates to the preparation of a nano-essential oil micelle solution using polyethylene glycol-b-polylactic acid (PEG-b-PLA) as a carrier.

### SUMMARY

In accordance with one broad aspect of the teachings described herein, a method of making an anti-microbial nano-particle containing an essential oil compound (EOC) may include the steps of:
a) mixing a quantity of an amphiphilic polymer with a quantity of a solvent comprising water to produce a suspension having a concentration of amphiphilic polymer that is less than about 40% wt;
b) heating the suspension to a processing temperature that is higher than a glass transition temperature of the amphiphilic polymer thereby formatting a plurality of polymeric micelles within the solvent, each micelle having a hydrophilic outer portion encasing a hydrophobic core and having a micelle diameter of less than about 80 nm;
c) adding a quantity of an essential oil (EOC) or components of such into the suspension so that a concentration of the essential oil compound is between about 0.2% and about 20% wt, whereby the EOC diffuses into and are encapsulated within the hydrophobic cores of each micelle.

Step c) may be performed after the plurality of micelles have been formed, and the EOCs may diffuse into the hydrophobic core of the micelles.

Step c) may be done at diffusion temperature of between about 20 degrees Celsius and about 99 degrees Celsius to promote diffusion of the EOC into the cores of the micelles.

The diffusion temperature may be between about 40 and about 70 degrees Celsius.

Step c) may include at least one of stirring and agitating the suspension to promote diffusion.

The at least one of stirring and agitating may be performed for a diffusion period that is between about 20 minutes and about 24 hours.

The stirring period may be between about 30 and about 120 minutes.

The at least one of stirring and agitating may be performed until the suspension is visually clear.

The method can include adjusting a pH of the suspension to between about 3 and about 9 whereby a hydrophilic portions of the amphiphilic polymer form salts having enhanced hydrophilicity.

The method may include crosslinking the micelles in the suspension whereby the nano-particle structure of the crosslinked micelles is preserved when the solvent is removed/evaporated.

The solvent may include at least one of an alcohol, sulphoxide, amine such that the solvent is miscible with water.

Step a) comprises providing the quantity of the amphiphilic polymer in a generally granular form in a container and then adding the quantity of solvent to the container.

The processing temperature may be at least 60 degrees Celsius, and/or may be at least 80 degrees Celsius.

The method may also include, after step c), cooling the suspension to less than about 30 degrees Celsius.

The amphiphilic polymer may include a sulfonated polyester.

The amphiphilic polymer may be a maleated polymer.

The essential oil compound may include at least one of thymol, carvacrol, eugenol, and limonene.

The essential oil compound comprises at least one of Thyme oil, Tea Tree oil, Pomegranate rind oil, Cinnamon leaf and oil of oregano.

The essential oil compound is added to the suspension in either a liquid or a solid state.

The quantity of the essential oil compound may be added gradually to the suspension over an introduction period that is between about 0.5 minutes and about 10 minutes.

The introduction period may be between about 1 and about 5 minutes.

A mass of the essential oil compound added in step c) may be substantially the same as a mass of the amphiphilic polymer provided in step a).

The plurality of polymeric micelles in the suspension after completing step b) may have a polydispersity index of (PDI) of less than about 40%.

Each micelle diameter may be less than about 50 nm or less than about 40 nm or less than about 30 nm.

In accordance with another broad aspect of the teachings described herein a polymeric micelle containing an essential oil compound may be formed using any of the methods described herein.

In accordance with another broad aspect of the teachings described herein a nano-particle may contain an essential oil compound (EOC). The nano-particle may include a micelle formed from amphiphilic polymer and may have a hydrophilic outer portion encasing a hydrophobic core comprising the essential oil compound and having a micelle diameter of less than about 80 nm.

The amphiphilic polymer may include at least one of a sulfonated polyester and a maleated polymer.

The essential oil compound may include at least one of thymol, carvacrol, eugenol, and limonene.

In accordance with another broad aspect of the teachings described herein, a method of applying an anti-microbial treatment to a fabric can include the steps of providing at least a first textile layer and applying a treatment solution onto the first textile layer. The treatment solution may include a plurality of nano-particles suspended in a solvent and each nano-particle may include a micelle formed from amphiphilic polymer and having a hydrophilic outer portion encasing a hydrophobic core comprising the essential oil compound and having a micelle diameter of less than about 80 nm. The method may also include evaporating the solvent whereby the plurality of nano-particles remain deposited on the first textile layer.

In accordance with another broad aspect of the teachings described herein, a fabric with anti-microbial properties can include at least a first textile layer and a plurality of nano-particles disposed on the first textile layer. Each nano-particle may include a micelle formed from amphiphilic polymer and having a hydrophilic outer portion encasing a hydrophobic core including the essential oil compound and having a micelle diameter of less than about 80 nm.

The amphiphilic polymer may include at least one of a sulfonated polyester and a maleated polymer.

The essential oil compound may include at least one of thymol, carvacrol, eugenol, and limonene.

In accordance with another broad aspect of the teachings described herein, a fabric with anti-microbial properties may include at least a first textile layer and a plurality of nano-particles disposed on the first textile layer. The plurality of nano-particles made by the methods described herein.

### BRIEF DESCRIPTION OF THE DRAWINGS

Embodiments of the present invention will be described with reference to the accompanying drawings, wherein like reference numerals denote like parts, and in which:

FIG. 1 is a flow chart showing one example of a method of making an anti-microbial nano-particle containing an essential oil compound (EOC);

FIG. 2 is a graph showing particle size measurement (hydrodynamic diameter, nm. analysis was performed on an Anton Paar Litesizer 100 with a 658 nm laser using Kalliope software);

FIG. 3 includes photos of one example of a suspension containing micelles with and without the presence of an anti-microbial agent;

FIG. 4 is a graph showing zones of inhibition of various particles prepared and placed on a plate; and

FIG. 5 is a schematic illustration of one example of a textile layer with nano-particles.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors or owners do not intend to abandon, disclaim, or dedicate to the public any such invention by its disclosure in this document.

Naturally occurring essential oil compounds (EOC) may be used as anti-microbial agents in a variety of applications, and may be relatively desirable because they are included in the GRAS (general regarded as safe) category by Food and Drug Authority of USA and placed in toxicity category IV for acute dermal and inhalation toxicity. Two factors influencing the use of EOC's are the delivery method and dosage control. EOC in relatively high concentrations can be irritating to the skin of a human user and the amount of an EOC that is applied to a surface using know techniques cannot be easily metered. A method to deliver measured and/or predictable doses of EOCs onto a surface or object would be an improvement in this area. As a first application such a solution and/or application technique could be the use of these antimicrobial particles on cloth surfaces to help inhibit microbe growth and allow for the extended use of clothing without laundering. This would be especially useful in hospital environments that have high exposure to microbes. Other applications could be surfaces that have high human traffic areas such as airplanes, hotels, public transit systems, restaurants etc.

One concept described in the present specification is the encapsulation of EOC's inside a particle which can then be applied to a target surface. This may be relatively advantageous in some circumstances because it may help provide an ability to control the release of the EOC from inside the particle by controlling the particle properties such as, for example, particle size or surface chemistry. Another possible benefit may be that the EOC may be relatively more protected from environmental factors such as oxygen and light than it would be if not encapsulated within a particle, which may help provide a relatively longer efficacy period after being applied to a surface. There are some known techniques to provide EOC's in particles but most processes to date result in particles having sizes that are greater than about 100 nm, and may tend to produce batches of particles that have a relatively wide polydispersity (PD) of particle sizes.

Furthermore, some of the known materials that can be used for form the particles are less desirable for use on human skin and/or on surfaces that may be touched by a human because they can be irritating and/or harmful in some biological applications. Therefore, there remains is a need to prepare EOC's in particles with small size less than 80 nm and preferably having a relatively narrow PD and being acceptably environmentally friendly to help facilitate the use of such particles in variety of scenarios.

In accordance with one broad aspect of the teachings described herein, a method for forming nano-particles as micelles that can contain the desired EOCs is described. Micelles as described herein are an aggregate of amphiphilic polymer and/or surfactant molecules dispersed in a solvent. The size of the micelle can be influenced by the relative size of the hydrophobic to hydrophilic groups of the given material or polymer used and the composition of the solvent system. Polymeric micelles can be particularly stable due to the fact that once formed the micelles do not tend to dissociate as easily as molecular micelles. Furthermore, the polymer outer portion can help shield the core of the micelle (and any compound contained therein) from external environmental factors and the molecular composition of the polymer may help regulate the release of the core component.

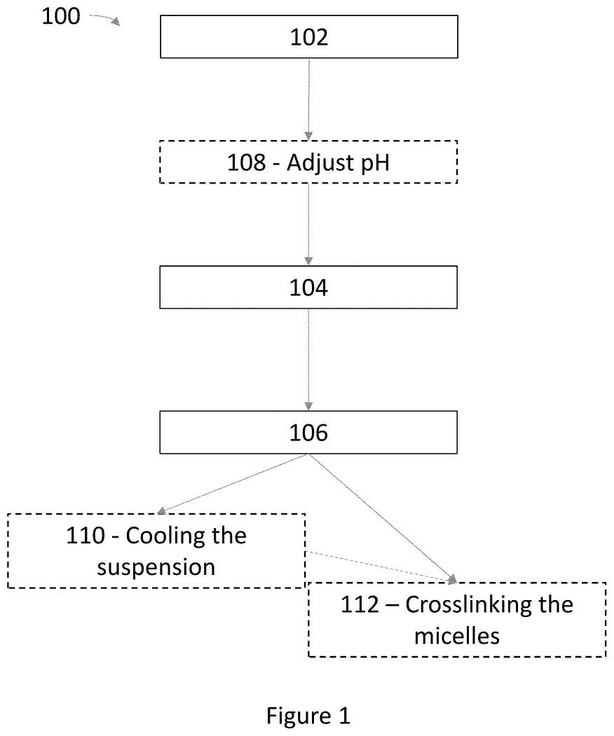

Referring to FIG. 1, one example of a method 100 of making an anti-microbial nano-particle containing an essential oil compound (EOC) includes, at step 102, mixing a quantity of an amphiphilic polymer with a quantity of a solvent (that can be entirely composed of water or may include at least some water with other compounds, such as suitable organic solvents, mixed therewith)) to produce a suspension having a target concentration of amphiphilic polymer.

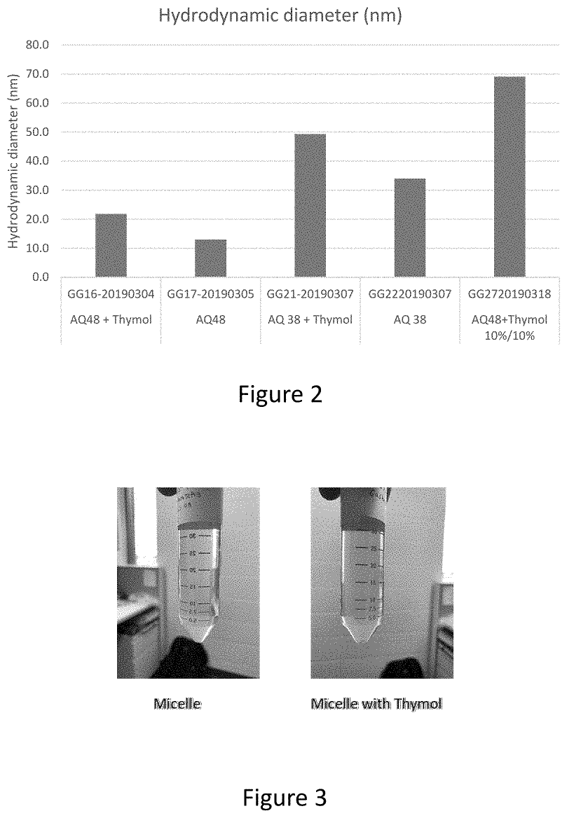

The amphiphilic polymer that is used in particles and methods described herein may be any suitable polymer including sulfonated polymers, a sulfonated polyester, polyethylene acrylic acid, polypropylene acrylic acid, maleated polymers, amine functionalized polymers and the like. One example of an environmentally friendly polymer that can form micelles in water is a sulfonated polyester (such as the Eastman AQ48, Eastman AQ49 and Eastman AQ55S polymers from the AQ™ series sold by Eastman Chemical as a food grade and cosmetic ingredient component). These materials may be generally safe for use on human skin or surfaces that a human may contact, as they are used in food and cosmetic production. These materials may also be relatively inexpensive as compared to some other polymers having analogous qualities, and when these tested polymer material were heated above their glass transition temperature (Tg—using the method described herein) it was demonstrated that they could form micellar particles having a desirable particle size, including particles having a diameter that is less than about 80 nm as shown in Table 1 and FIG. 2.

| | | Polymer | | EO | | |
|---|---|---|---|---|---|---|
| Sample | | Solids | Essential | solids | Size | PDI |
| # | Polymer | Content | Oil | Content | (nm) | (%) |
| GG17 | Eastman AQ48 | 5% | none | | 13 | 26.6 |
| GG16 | Eastman AQ48 | 5% | Carvacrol | 3% | 21.8 | 21% |
| GG27 | Eastman AQ49 | 10% | Carvacrol | 10% | 69.1 | 30% |
| GG22 | Eastman AQ55S | 5% | none | | 33.9 | 27.8 |
| GG21 | Eastman AQ55S | 5% | Thymol | 3% | 49.3 | 27.6 |

The polymer may be introduced into the solvent in any suitable form, including solid and liquid forms. Preferably, the amphiphilic polymer may be provided in a generally granular form. The amphiphilic polymer can, as part of step 102, be held in a suitable container or vessel and the solvent liquid can then be added into the container—optionally while stirring or otherwise agitating the mixture to help dissolve the amphiphilic polymer.

The solvent used in step 102 can be any suitable solvent that can dissolve the desired amphiphilic polymer, and preferably may be generally safe for use on and around humans and human skin, as well as on surfaces or objects that may be contacted by a human. This may allow the suspension to be used to treat clothing, furniture, counters and similar surfaces, desks, vehicle interiors and the like. In the examples described herein, and as tested, the solvent was water, and preferably distilled water that was substantially free of other impurities. Other types of water may be used in other examples. Optionally, one or more other liquids may be added to the solvent to help modify the solvent's properties. For example, additives may be used to help alter the solubility of the amphiphilic polymer in the solvent (preferably to enhance its solubility), alter the suspension's pH, impart a fragrance or for other such purposes. These additives may be any suitable substance, and may include alcohols, amines, sulphoxides and the like. In other applications, where human interaction is not likely it is possible that different solvents may be used.

In some examples, the solvent may have a pH that is outside a desired, target pH range. For example, compounds that are added to the water when preparing the solvent may cause the pH to become more acidic, or more basic, than is desired to help facilitate the dissolving of the amphiphilic polymer and/or the formation of the desired micelles. In such examples, the method may include the optional step 108 of adjusting the pH of the suspension to between about 3 and about 11 (and preferably between 3 and 9) whereby, in some examples the hydrophilic portions of the amphiphilic polymer may form salts that may have enhanced hydrophilicity. This may be done by adding other compounds, such as acids or bases into the suspension.

The quantity of the amphiphilic polymer and solvent can be selected to provide a desired concentration of the amphiphilic polymer in the resulting suspension. The target or desired concentration of the amphiphilic polymer can be less than about 40% wt, about 30% wt, about 20%, about 10% wt and about 5% wt. The target concentration for a given suspension may be influenced by the desired end use of the suspension. For example, suspensions that are intended to be sprayed onto fabrics, or related porous retentive surfaces, or the like the target concentration may be between about 1% wt and about 5% wt, and may be about 2-5% wt. Alternatively, if the suspension is intended to be used to form a polymeric film on a surface the target concentration may be between about 5% wt and about 20% wt, less than about 10% and may be about 5% or less.

Having formed the desired suspension in step 102, the method can advance to step 104 that includes heating the suspension to a processing temperature that is higher than a glass transition temperature of the amphiphilic polymer. The processing temperature in a given example of this method may depend on the glass temperature of the specific amphiphilic polymer used in that example, and preferably the amphiphilic polymer is selected so that its glass temperature is less than the boiling temperature of the solvent—under the conditions in which step 104 is carried out. For example, if step 104 is conducted at generally atmospheric pressure then the amphiphilic polymer can be selected to have glass temperature that is less than 100 degrees Celsius. This may help reduce the chances of boiling the water-based solvent during this step if conducted at atmospheric pressure. Alternatively, if step 104 is conducted at an elevated pressure it may be possible to heat a water-based solvent above 100 degrees Celsius without boiling. In other examples some amount of solvent loss (to boiling or otherwise) may be acceptable during step 104, provided the concentration of the amphiphilic polymer remains within the desired concentration ranges. It may also be desirable for the processing temperature to be above a minimum threshold as the temperature of the suspension may affect the rate at which the amphiphilic polymer is dissolved. For example, the processing temperature may be configured to be at least 60 degrees Celsius, at least 70 degrees Celsius, at least 80 degrees Celsius, at least 90 degrees Celsius or more.

When above their glass temperature, the molecules of the amphiphilic polymer can rearrange themselves within the solution to form a plurality of hollow nano-particles in the form of polymeric micelles, each having a hydrophilic outer portion or shell that surrounds a hydrophobic core or interior region. This core can be at least partially hollow such that other molecules can be encased within the core of each micelle. Preferably, the method is configured (e.g. the polymer and solvent materials are selected and processed at the suitable temperatures) so that the diameters of the micelles formed are less than about 80 nm, and preferably less than about 70 nm, less than about 60 nm, less than about 50 nm, less than about 40 nm, less than about 30 nm and may be about 20 nm.

Preferably, the polymeric micelles formed using the described methods can have a polydispersity index (PDI) that is within a target range, and preferably is less than about 40%. The PDI as described herein is a measure of the narrowness of the particle size dispersion, and can be calculated using any suitable technique. Having a PDI that is less than about 40% may help provide micelles of generally similar size, which may make the particles in the suspension relatively more homogenous and may help ensure that the active effect of the solution is generally the same for a given applied volume of the suspension. That is, if a film is created using the suspension having micelles with the target PDI then different regions of the film should contain micelles of approximately the same size and may therefore exhibit similar properties in terms of efficacy, active shelf life and the like. Optionally, the suspension may be formed such that it has a PDI that is less than about 40%, less than about 35%, less than about 30%, less than about 25% and may be about 20%.

The method 100 can then proceed to step 106 in which a quantity of any suitable essential oil compound (EOC) or components of such into the suspension that contains the plurality of formed, polymeric micelles in the solvent. The EOC is preferably selected to have desired anti-microbial properties, while being generally safe for use with human skin or objects that will be in contact with humans. Some examples of suitable EOCs include thymol, carvacrol, eugenol, limonene, thyme oil, tea tree oil, pomegranate rind oil, cinnamon leaf and oil of oregano. Other related compounds may also be used.

The EOC is preferably added into the suspension until the concentration of the EOC reaches a predetermined target threshold, which may be between about between about 0.2% and about 20% wt, and may be between about Optionally, the amount (e.g. mass) of the EOC that is added can be approximately the same as the mass of the amphiphilic polymer that is included in a given suspension (e.g. as was used in step 102).

The EOC is preferably added to the solution after the micelles have been formed in step 104. In this arrangement, the EOC molecules can disperse through the suspension (optionally with the assistance of mechanical stirring and/or agitation) and can diffuse through the outer shells of the micelles and can move into and become encapsulated within the cores of each micelle. Alternatively, at least some of the EOC's may be added to the suspension while the amphiphilic polymer is being introduced and/or while the micelles are being formed.

At the end of this step 106, the suspension may include a plurality of polymeric micelles that each contain a small amount/dose of the EOC compound. This EOC dose can remain contained within the micelle until the micelle is ruptured or otherwise disassembles.

To help promote the diffusion of the EOC within the suspension, the suspension may be maintained at a diffusion temperature while the EOC is being added and for the duration of a diffusion period that follows.

The diffusion temperature is preferably below the boiling temperature of the suspension (at its reaction pressure), and may be between about 20 degrees Celsius and about 99 degrees Celsius, or between about 40 degrees Celsius and about 70 degrees Celsius if this step 106 is conducted at about atmospheric pressure. The diffusion temperature may be held generally constant during the diffusion period, or alternatively, the diffusion temperature may be changed during the diffusion period while generally staying within the upper and lower limits of the diffusion temperature ranges.

The diffusion period (e.g. the amount of time it takes to perform step 106) can be any suitable time that is sufficient to allow a desired portion of the EOC to migrate into the cores of the micelles. In the examples described herein, the diffusion period may be between about 20 and about 24 hours or more, or may be between about 30 and about 120 minutes. In some examples, the diffusion period can be continued until a sufficient amount of the EOC has been capture within the micelles such that the suspension becomes visually clear which can indicate the substantially all of the EOCs have migrated into the cores of the micelles (having been more opaque when the EOC was first introduced). Optionally, the suspension can be stirred or otherwise agitating during the diffusion period to help promote the diffusion of the EOC.

The EOCs added in this step 106 may be added in any desired state, including as a liquid or in a solid or crystalline state. In some examples, it may be desirable to add the EOCs gradually to the suspension, rather than in one single dose. This may help in minimizing exposure of EOC to temperature and oxygen. In such cases, the EOCs can be added over the course of an introduction period that can be any suitable length of time, any may be between about 30 seconds (0.5 minutes) and about 10 minutes or more, or may be between about 1 minute and about 5 minutes.

Optionally, after completing step 106 the method can include optional step 110 in which the suspension is then cooled from the diffusion temperature to a storage or use temperature that is lower, and may be less than about 30 degrees Celsius or less than about 20 degrees Celsius.

In some embodiments of the teachings described herein, such as if the intended use of the suspension is to form a film on a surface, the method may, in some instances include the optional step 112 crosslinking the polymeric micelles while in the suspension. Such crosslinking may help preserve the generally individual, nano-particle structure of the micelles when the solvent is removed (or at least partially removed). This may help facilitate forming a polymeric film that includes the micelles in which the individual micelles remain discrete and generally intact nano-particles as the solvent evaporates and the film is formed, whereby the EOCs remain contained within the micelles (instead of the EOCs being released and simply diffusing within the polymer matrix as the film is formed). This may help extend the useful life of the EOCs within the film and allow them to be released over an extended period of time. Experiments have been conducted in which micelles containing a sample EOC were created using the methods described herein. As can be seen in FIG. 2 the inclusion of the anti-microbial moiety (for the thymol example) increases the micelle size from 11 nm to about 22 nm. The small particle size can be qualitatively seen in FIG. 3 where a solution of the micelle and the anti-microbial containing micelle appear to be virtually clear. These particles were then used as is in the anti-microbial testing protocol to test their efficacy.

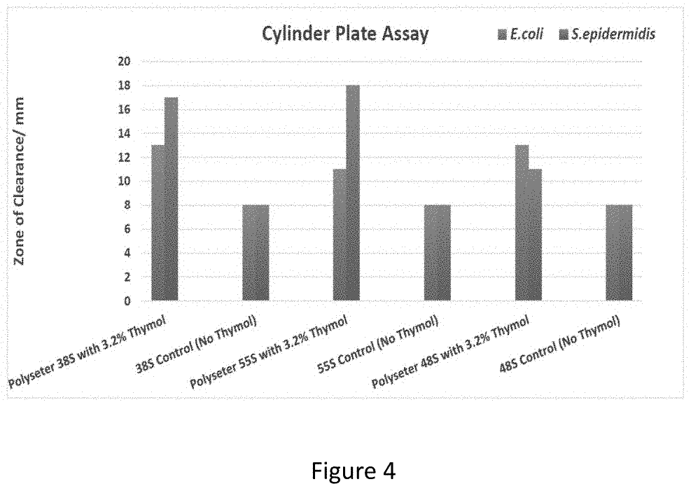

In these experiments, the nano-particles were tested by using an assay procedure whereby an agar plate was grown with different bacteria (E. coli and S. epiderimidis) to enable a homogenously dispersed lawn to form on the surface. The particles were deposited onto the bacteria and the diameter of the zone of inhibition was measured. This is the region where the bacteria are killed (bactericidal), or prevented from multiplying (bacteriostatic) by the diffusion and hence migration of the anti-microbial moiety from within the particle onto the plate and surrounding area. The larger the zone of inhibition the more effective the anti-microbial moiety. FIG. 4 shows the zone of inhibition of various particles prepared (see experimental section) and placed on the plate. Note that controls are included which contain no encapsulated moieties and show that the anti-microbial containing particles do indeed inhibit, or render bacteria incapable of propagation.

This concept of using a hydrophobic anti-microbial moiety that migrates into the core of the micelle was further tested by using other potential materials such as Eugenol, Limonene and Pomegranate rind extracted oils (Table 2) and encapsulating them in the sulfonated polyester.

TABLE 2

| | | anti-microbial | PE/AM |
|---|---|---|---|
| Experiment | Polyester | (AM) | (g/g) 1 |
| GC16-20190304 | Eastman AQ48 | carvacrol | 2.5/1.5 |
| GC17-20190305 | Eastman AQ48 | none | 2.5/0 |
| GG18-20190307 | Eastman AQ48 | carvacrol | 2.5/1.5 |
| GG19-20190307 | Eastman AQ38S | Thymol | 2.5/1.5 |
| GG20-20190307 | Eastman AQ38S | none | 2.5/0 |
| GG21-20190307 | Eastman AQ55S | Thymol | 2.5/1.5 |
| GG22-20190307 | Eastman AQ55S | none | 2.5/0 |
| GG23-20190312 | Eastman AQ48 | Thymol | 2.5/1.5 |
| GG24-20190312 | Eastman AQ48 | none | 2.5/0 |
| GG27-20190318 | Eastman AQ48 | Thymol | 10/10 |
| GG28-20190402 | Eastman AQ38S | Limonene | 2.5/1.5 |
| GG29-20190402 | Eastman AQ38S | Eugenol | 2.5/1.5 |
| GG30-20190402 | Eastman AQ38S | Thymol | 2.5/1.5 |
| GG31-20190402 | Eastman AQ38S | Thymol | 2.5/1.5 |
| GG34-20190418 | Eastman AQ48 | Eugenol | 2.5/1.5 |
| GG35-20190418 | Eastman AQ48 | Limonene | 2.5/1.5 |
| GG36-20190424 | Eastman AQ48 | Eugenol | 5/5 |
| GG37-20190430 | Eastman AQ48 | Pomegranate | 2.5/5 ² |

1. 5% solutions of PE.
² 5 g of pomegranate extract solution

Representative details of some of these experiments are summarized below, for which the materials were purchased from Aldrich chemical company while the sulfopolyesters were Eastman AQ™, from Eastman Chemical.

### GG16-20190304

In a beaker was added Polyester AQ48 (Eastman Kodak, 2.5 g) and then distilled water (47.5 g). This was heated to a processing temperature of about 60° C. at which point the polyester formed micelles resulting in a clear solution. To this was added dropwise over two minute's cavracrol (3 g), and stirred for two hours resulting in a generally visually clear solution. This was then cooled to room temperature and for anti-microbial testing.

### GG23-20190312

In a beaker was added Polyester AQ48 (Eastman Kodak, 2.5 g) and then distilled water (47.5 g). This was heated to a processing temperature of about 60° C. at which point the polyester formed micelles resulting in a clear solution. To this was added dropwise over two minute's Thymol (3 g), and stirred for two hours resulting in a clear slightly brownish coloured solution. This was cooled to room temperature and used as is for anti-microbial testing.

While some examples of microbes have been described herein, the microbes that may be treated using the EOC containing nano-particles may include Gram positive and Gram negative bacteria, archaebacteria, enveloped and non-enveloped viruses, fungi including mold and yeasts, bacterial endospores and the like.

Another possible application of the essential oil-containing nano-particles described herein is in the deposition of the essential oil-containing nano-particles on a surface, cloth or other similar substrate to help impart at least some degree of anti-microbial properties to the treated substrate. For example, the essential oil-containing nano-particles may be suspended in a polymer or other analogous type of material that can be applied as a film or other similar coating to a surface, such as a hand rail or a countertop. When the film is deposited, the essential oil-containing nano-particles are held within the film and as people or objects come into contact with the film-coated surface microbes that are deposited on the substrate can contact the essential oil-containing nano-particles. This can help provide a time-release or relatively long lasting type of anti-microbial protection for a treated/coated substrate.

In another example, the essential oil-containing nano-particles may be used to treat cloth and/or fabric to impart anti-microbial properties to the treated fabric. The essential oil-containing nano-particles may be suspended in a suitable solvent and can be applied to the fabric (such as by misting, spraying, soaking or the like). The solvent can evaporate to leave a generally dry fabric in which a plurality of essential oil-containing nano-particles are embedded. When contacted with when the fabric is in use, the contained essential oil can thereby kill at least some of the microbes present on the fabric. Since the essential oils are encapsulated additional amounts of the essential oil compounds will be released over time which can help make the anti-microbial effect last for at least a portion of the time when the fabric is in use.

For example, one method of applying an anti-microbial treatment to a fabric can include the steps of providing at least a first textile layer. A given fabric may have two or more textile layers, and each layer may be of any suitable configuration (such as being woven or non-woven) and can be made from natural or synthetic materials or a blend thereof.

To impart the desired anti-microbial properties the essential oil-containing nano-particles can be deposited onto a least one layer of the fabric (e.g. the first textile layer) using any suitable technique, such as applying a treatment solution onto the first textile layer. The treatment solution preferably includes a plurality of nano-particles suspended in a solvent (such as water), and at least some of the nano-particles include a micelle formed from amphiphilic polymer and having a hydrophilic outer portion encasing a hydrophobic core comprising the essential oil compound and having a micelle diameter of less than about 80 nm—and may include any of the other properties described herein.

This application of the solution to the fabric can be done by spraying/misting the fabric with the solution, by submerging the fabric in a vessel containing the solution or via other suitable methods. After the solution has been applied, the treating method can include the step of evaporating at least some of the solvent whereby the plurality of nano-particles remain deposited on the first textile layer in a desired concentration.

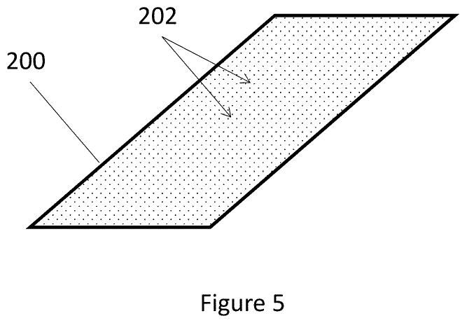

Using these methods, or other similar methods, can allow for the preparation of a fabric with anti-microbial properties, such as schematically illustrated in FIG. 5, that includes at least the first textile layer 200 that is treated so that it has a plurality of nano-particles 202 disposed on the first textile layer. The nano-particles 202 can be any of the particles described herein, and preferably include a micelle formed from amphiphilic polymer and having a hydrophilic outer portion encasing a hydrophobic core comprising the essential oil compound and having a micelle diameter of less than about 80 nm. While shown as being generally homogeneously distrusted across the textile layer 200 in this example, the particles 202 can have other distributions in other examples.

To help evaluate the effectiveness of the EOC containing nano-particles described herein at killing microbes, including viruses and bacteria, when introduced on a cloth testing was conducted, the results of which are summarized herein. While the test results described below are related to the behaviour of the EOC containing nano-particles on a cloth substrate, the inventors believe that similar results will be obtained if the EOC containing nano-particles are brought into contact with microbes in other circumstances such as being included in a film or otherwise deposited on to a non-cloth surface and in other such applications. That is, the inventors believe, based at least in part on the results discussed below, that the EOC containing nano-particles are stable enough to remain viable when deposited on a surface or cloth or if included within film, surface coating, or other such carrier until they contact a target microbe.

For example, in one series of tests to quantitate the effectiveness of the EOC containing nano-particles for treating viruses, tests were conducted to quantitate live virus after treatment with EOC containing nano-particles (also referred to as EOiP in the tables herein) using a plaque assay technique. The plaque assay is based on the concept of bacteriophages infecting the bacterial host and as the phage replicates inside the cell, causing the cell to rupture and release new virions. The new phages infect nearby cells and the process is repeated. This creates a clear zone on a lawn of bacteria and that zone is counted as one plaque, reflecting that one virus started the process. Using this assay, the number of live viruses that were initially present in the sample can be calculated. The bacteriophage Phi6 was used in these tests because it is a lipid coated (enveloped) dsRNA virus which belongs to the broad family of bacteriophages, Cystoviridae. The enveloped virions are spherical, about 85 nm in diameter and covered by spikes. This enveloped structure surrounds an isometric nucleocapsid which is about 58 nm in diameterix. Phi6 has a very similar morphology to enveloped human viral pathogens like COVID-19 and Influenza. Hence, it was used for experimentation along with Pseudomonas syringae, which was the host bacteria for phi6.

In these tests, the phi6 virus surrogate system was tested using a cloth testing protocol. In this example, a dispersion of the EOC containing nano-particles were introduced onto cloth as a spray (e.g. a treatment solution) and then the cloth was allowed to dry from about 1 minutes to about 1 hour prior to viral load applications to help allow at least some of the solvent in the spray to evaporate. Subsequently the Phi6 was applied to the cloths for time intervals ranging from 1, 5, 10, 30 and 60 minutes. The cloth was then vortexed in a water solution and then plated onto the bacterial plates and incubated to see how much virus was alive. Various Essential oils at different concentrations were tested and the results are shown in table 3, which contains results from testing of EOC containing nano-particles, rosemary, and limonene and negative control using water at a pH of 4.5 or lower using phi6. Log₁₀ reduction of the viral load was calculated by comparing the results obtained for each sample to a negative control developed using water.

TABLE 3

| | Amount of | | | | | |
|---|---|---|---|---|---|---|
| | EOiP applied | | Average | Average | Percent | Log₁₀ |
| | to test | Exposure | PFU/cloth | PFU/cloth | reduction | reduction |
| | swatches | time | (PFU/mL) | (PFU/mL) | compared to | compared to |
| Sample | (μL) | (Minutes) | Sample | Control | control | control |
| Rosemary 2% | 50 | 60 | 6.33 × 10⁴ | 5.50 × 10⁶ | 98.8 | 1.94 |
| (GG1-BK133) | | | <1 × 10⁰ | 5.00 × 10⁸ | 100 | >3.70 |
| PL137B | | | <1 × 10⁰ | 5.00 × 10⁸ | 100 | >3.70 |
| (2% rosemary) | | | | | | |
| PL137C | | | <1 × 10⁰ | 5.00 × 10⁸ | 100 | >3.70 |
| (2% rosemary) | | | | | | |
| Limonene 3% | 50 | 60 | 3.27 × 10⁴ | 5.0 × 10⁶ | 99.4 | 2.23 |
| (GG1-BK136) | | | <1 × 10⁰ | 5.00 × 10⁸ | 100 | >3.70 |

It was also found that when the essential oil was Thymol, significantly higher Log reductions were achieved even after only minute's exposure to the EOC containing nano-particles as shown in table 4, which shows testing of thymol+5% AQ48 and negative control using water at a pH of 4.5 or lower using phi6. Log₁₀ reduction of the viral load was calculated by comparing the results obtained for each sample to a negative control developed using water.

TABLE 4

| | Amount of | | | | | |
|---|---|---|---|---|---|---|
| | EOiP applied | | Average | Average | Percent | Log₁₀ |
| | to test | Exposure | PFU/cloth | PFU/cloth | reduction | reduction |
| | swatches | time | (PFU/mL) | (PFU/mL) | compared to | compared to |
| Sample | (μL) | (minutes) | Sample | Control | control | control |
| Thymol 3.2% | 10 | 5 | 0.33 × 10² | 3.84 × 10⁸ | 99.99991 | 6.07 |
| (GG1-BK23) | 50 | 1 | <1.0 × 10⁰ | 5.73 × 10⁸ | 100 | >5.75 |
| | 50 | 10 | <1.0 × 10⁰ | 3.20 × 10⁸ | 100 | >5.5 |
| | 50 | 1 | <1.0 × 10⁰ | 1.20 × 10⁸ | 100 | >5.08 |
| | 50 | 5 | <1.0 × 10⁰ | 6.95 × 10⁷ | 100 | >4.84 |
| GG2-BK57 | 10 | 5 | <1.0 · 10⁰ | 8.41 × 10⁸ | 100 | >5.92 |
| (4% Thymol) | | | | | | |

In addition to anti-viral testing, tests were conducted to determine the effectiveness of the antibacterial properties of the EOC containing nano-particles. Specifically, to demonstrate the antibacterial efficacies of the EOC containing nano-particles formulation an antibacterial efficiency assay was performed. The bacterial species is exposed to the antimicrobial substance and the degree of inactivation of the bacteria is recorded. Three bacteria were tested; Salmonella choleraesuis, Staphylococcus aureus and Pseudomonas aeruginosa.

To quantify the degree of bacterial inactivation by EOC containing nano-particles and to reflect the real-world scenario of the treatment this experimentation was performed using cotton cloth swatches as a representative of porous surfaces. In the procedure a pre-determined amount of EOC containing nano-particles is loaded on the cloth and was dried for 2 hours. After 2 hours, the cloth was loaded with bacteria and allowed to sit for a selected period. Once the exposure was complete, the cloth was added to a definite volume of water and vortexed to recover all the bacteria from the cloth. Following this, the sample was serially diluted appropriately and then plated.

This method of experimentation was designed to reflect a situation where bacteria that lands on any porous surface that has been sprayed with the EOP formulation gets inactivated.

Tables 5, 6 and 7 show the reduction of Salmonella choleraesuis, Staphylococcus aureus and Pseudomonas aeruginosa, respectively and greater than log 4 reduction was attained except for the P. aeruginosa. Furthermore it can be seen that even at low EOiP dosages and short times of 10 minutes, there was sufficient anti-microbial activity to generate Log 4 reduction in bacterial count for two of the three bacteria.

TABLE 5

| | Amount of | | | | |
|---|---|---|---|---|---|
| | EOP applied | | Average | Average | Percent |
| | to test | Exposure | CFU/cloth | CFU/cloth | reduction |
| | swatches | time | (CFU/mL) | (CFU/mL) | compared to |
| Sample | (μL) | (Minutes) | Control | Sample | control |
| Thymol | 50 | 10 | 1.49 × 10⁷ | <1.0 × 10⁰ | 100 |
| 3.2% | | | | | |
| Thymol | 10 | 10 | 1.96 × 10⁷ | 6.67 × 10² | 99.997 |
| 3.2% | | | | | |
| Thymol | 5 | 10 | 1.89 × 10⁷ | 5.53 × 10⁴ | 99.7 |
| 3.2% | | | | | |

Table 5 shows the efficacy of EOP solutions against S. choleraesuis using thymol (3.2% EO, 5% AQ48), Samples were compared to a negative control developed using water. Tests were performed at pH 4.5.

TABLE 6

| | Amount of | | | | |
|---|---|---|---|---|---|
| | EOP applied | | Average | Average | Percent |
| | to test | Exposure | CFU/cloth | CFU/cloth | reduction |
| | swatches | time | swatch | swatch | compared to |
| Sample | (μL) | (Minutes) | (Control) | (Sample) | control |
| Thymol | 50 | 10 | 7.93 × 10⁶ | 1.0 × 10⁰ | 100 |
| 3.2% | | | | | |
| Thymol | 10 | 10 | 1.24 × 10⁹ | 4.23 × 10⁴ | 99.997 |
| 3.2% | | | | | |
| Thymol | 5 | 10 | 1.43 × 10⁹ | 3.40 × 10⁶ | 99.8 |
| 3.2% | | | | | |

Table 6 shows the efficacy of EOP solutions against S. aureus using thymol (3.2% EO, 5% AQ48), limonene (3% EO, 5% AQ48), rosemary (2% EO, 5% AQ48) and 5% AQ48 without EO. Samples were compared to a negative control developed using water. Tests were performed at pH 4.5.

TABLE 7

| | Amount of | | | | | |
|---|---|---|---|---|---|---|
| | EOP applied | | Average | Average | Percent | Log₁₀ |
| | to test | Exposure | CFU/cloth | CFU/cloth | reduction | reduction |
| | swatches | time | (CFU/mL) | (CFU/mL) | compared to | compared to |
| Sample | (μL) | (Minutes) | (Control) | (Sample) | control | control |
| Thymol 3.2% | 10 | 10 | 4.92 × 10⁹ | 1.79 × 10⁷ | 99.6 | 2.44 |
| Thymol 4% | 10 | | 1.33 × 10⁸ | 2.56 × 10⁶ | 98.1 | 1.71 |

Table 7 shows the efficacy of thymol EOP solutions (1.6% EO+5% AQ48 and 4% EO+5% AQ48) and 5% AQ48 without any EO against the test organism P. aeruginosa. A negative control was developed using water at the respective pH of the sample (4.5). Samples were compared to a negative control developed using water. Tests were performed at pH 4.5.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent, or patent application was specifically and individually indicated to be incorporated by reference in its entirety.

We claim:

1. A method of making an anti-microbial nano-particle containing an essential oil compound (EOC), the method comprising:
a) mixing a quantity of an amphiphilic polymer with a quantity of a solvent comprising water to produce a suspension having a concentration of the amphiphilic polymer that is less than about 40% wt;
b) heating the suspension to a processing temperature that is higher than a glass transition temperature of the amphiphilic polymer thereby formatting a plurality of polymeric micelles within the solvent, each micelle having a hydrophilic outer portion encasing a hydrophobic core and having a micelle diameter of less than about 80 nm;
c) adding a quantity of an essential oil (EOC) or components of such into the suspension so that a concentration of the essential oil compound is between about 0.2% and about 20% wt, whereby the EOC diffuses into and are encapsulated within the hydrophobic cores of each micelle.

2. The method of claim 1, wherein step c) is performed after the plurality of micelles have been formed, and wherein the EOCs diffuse into the hydrophobic core of the micelles.

3. The method of claim 1, wherein step c) done at diffusion temperature of between about 20 degrees Celsius and about 99 degrees Celsius to promote diffusion of the EOC into the cores of the micelles.

4. The method of claim 3, wherein the diffusion temperature is between about 40 and about 70 degrees Celsius.

5. The method of claim 1, wherein step c) further comprises at least one of stirring and agitating the suspension to promote diffusion.

6. The method of claim 5, wherein the at least one of stirring and agitating is performed for a diffusion period that is between about 20 minutes and about 24 hours.

7. (canceled)

8. (canceled)

9. The method of claim 1, further comprising adjusting a pH of the suspension to between about 3 and about 9 whereby a hydrophilic portions of the amphiphilic polymer form salts having enhanced hydrophilicity.

10. The method of claim 1, further comprising crosslinking the micelles in the suspension whereby the nano-particle structure of the crosslinked micelles is preserved when the solvent is removed/evaporated.

11. The method of claim 1, wherein the solvent further comprises at least one of an alcohol, a sulphoxide, and an amine such that the solvent is miscible with water.

12. The method of claim 1, wherein step a) comprises providing the quantity of the amphiphilic polymer in a generally granular form in a container and then adding the quantity of solvent to the container.

13. (canceled)

14. (canceled)

15. (canceled)

16. (canceled)

17. (canceled)

18. The method of claim 1, wherein the essential oil compound comprises at least one of thymol, carvacrol, eugenol, and limonene.

19. (canceled)

20. (canceled)

21. The method of claim 1, wherein the quantity of the essential oil compound is added gradually to the suspension over an introduction period that is between about 0.5 minutes and about 10 minutes.

22. (canceled)

23. The method of claim 1, wherein a mass of the essential oil compound added in step c) is substantially the same as a mass of the amphiphilic polymer provided in step a).

24. The method of claim 1, wherein the plurality of polymeric micelles in the suspension after completing step b) have a polydispersity index of (PDI) of less than about 40%.

25. The method of claim 1, wherein each micelle diameter is less than about 50 nm or less than about nm or less than about 30 nm.

26. (canceled)

27. A nano-particle containing an essential oil compound (EOC), the nano-particle comprising a micelle formed from amphiphilic polymer and having a hydrophilic outer portion encasing a hydrophobic core comprising the essential oil compound and having a micelle diameter of less than about 80 nm.

28. The nano-particle of claim 27, wherein the amphiphilic polymer comprises at least one of a sulfonated polyester and a maleated polymer.

29. The nano-particle of claim 28, wherein the essential oil compound comprises at least one of thymol, carvacrol, eugenol, and limonene.

30. A method of applying an anti-microbial treatment to a fabric, the method complying:
a. providing at least a first textile layer;
b. applying a treatment solution onto the first textile layer, the treatment solution comprising a plurality of nano-particles suspended in a solvent and each nano-particle comprising a micelle formed from amphiphilic polymer and having a hydrophilic outer portion encasing a hydrophobic core comprising the essential oil compound and having a micelle diameter of less than about 80 nm;
c. evaporating the solvent whereby the plurality of nano-particles remain deposited on the first textile layer.

31. (canceled)

32. (canceled)

33. (canceled)

34. A fabric with anti-microbial properties, the fabric comprising:
a. at least a first textile layer; and
b. a plurality of nano-particles disposed on the first textile layer, the plurality of nano-particles made by the method of claim 1.
